(19) United States
(12) Patent Application Publication
(10) Pub. No.: US 2021/0169439 A1
(43) Pub. Date: Jun. 10, 2021

(54) ESOPHAGEAL STETHOSCOPE

(71) Applicants: THE ASAN FOUNDATION, Seoul (KR); UNIVERSITY OF ULSAN FOUNDATION FOR INDUSTRY COOPERATION, Ulsan (KR)

(72) Inventors: Sung Hoon KIM, Seoul (KR); Young Jin MOON, Guri-si (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 17/183,145

(22) Filed: Feb. 23, 2021

Related U.S. Application Data
(63) Continuation of application No. PCT/KR2019/014491, filed on Oct. 30, 2019.

(30) Foreign Application Priority Data
Oct. 30, 2018 (KR) 10-2018-0130471
Oct. 30, 2019 (KR) 10-2019-0136240

(51) Int. Cl. A61B 7/02; A61B 7/04; A61B 5/0205; A61B 5/00

(52) U.S. Cl. CPC A61B 7/023; A61B 7/04; A61B 7/026; A61B 5/6853; A61B 5/02055

(57) ABSTRACT

An esophageal stethoscope is disclosed. The esophageal stethoscope includes a cuff, a tube, one end of which is disposed in the interior of the cuff, and extending to a side that is opposite to the one end thereof, a mount member mounted on an opposite end of the tube, and a microphone disposed in the interior of the mount member, the tube includes at least one hole configured to allow sound waves that passes through the cuff to be provided into the tube, and the microphone absorbs cardiac sound and converts the cardiac sound to a cardiac sound electric signal, and is electrically connected to an external device to deliver the cardiac sound electric signal to the external device.

### CROSS-REFERENCE TO RELATED APPLICATIONS

The present application is a continuation of International Patent Application No. PCT/KR2019/014491, filed on Oct. 30, 2019, which is based upon and claims the benefit of priority to Korean Patent Application No. 10-2018-0130471 filed on Oct. 30, 2018, and Korean Patent Application No. 10-2019-0136240 filed on Oct. 30, 2019. The disclosures of the above-listed applications are hereby incorporated by reference herein in their entirety.

### BACKGROUND

The inventive concept relates to an esophageal stethoscope, and more particularly, relates to an esophageal stethoscope by which the state of a patient can be identified in a non-invasive manner by using cardiac sound or lung sound.

A vital sign is a mechanism of a change in the body, which occurs through the body temperature, the pulse, the respiration, and the blood pressure of a patient. The vital sign is measured before and after a surgical operation, during a surgical operation, before and after a dangerous diagnostic inspection, before and after injection of a medicine that influences a heart/blood relationship or a respiratory function, and the like, and takes a very important role in monitoring the health state of a patient.

Methods for measuring a vital sign include an invasive method and a non-invasive method. The invasive method is a method including an invasion process of a needle or the like passing through a portion of a body tissue, and a non-invasive method is a method that does not include an invasion process performed in the invasive method. The invasive method has an advantage of allowing a more precise inspection, but has a disadvantage of causing a damage to a blood vessel, a bruise, an infection, and the like in the invasion process.

The esophageal stethoscope is one of devices that may monitor a vital sign of a patient before and after a surgical operation or during a surgical operation in a non-invasive method. The esophageal stethoscope is a medical detection device that is inserted into the throat of a patient to detect cardiac sound or lung sound. In particular, the esophageal stethoscope is useful when cardiac sound or lung sound of a patient (an anesthesia patient or a critical patient) has to be heard in real time before and after a surgical operation, or during a surgical operation.

The conventional esophageal stethoscope includes a configuration that is inserted into a throat to absorb cardiac sound and lung sound, a tube that delivers the absorbed cardiac sound and lung sound, and a stethoscope part that allows the delivered cardiac sound and lung sound to be heard. Accordingly, the conventional esophageal stethoscope is used in a manner in which a tube is inserted into the body of a patient through the throat of the patient, and a stethoscope is directly mounted on ears of a user, and cardiac sound and lung sound delivered through the tube are heard by the user. Accordingly, the user has to intuitively determine information on the cardiac sound and the lung sound, and it is difficult to utilize the acquired cardiac sound and lung sound through the esophageal stethoscope as effective information.

Furthermore, in the conventional esophageal stethoscope, the sound quality is decreased for the reason that the intensity of sound waves becomes weaker in a process of delivering the cardiac sound and the lung sound and noise is contained in the sound waves.

### SUMMARY

Embodiments of the inventive concept provide an esophageal stethoscope that may allow cardiac sound and lung sound, which are obtained in the interior of the body of a patient, to be visually identified through an electric signal obtained through conversion without hearing the cardiac sound and the lung sound through a stethoscope.

Embodiments of the inventive concept also provide an esophageal stethoscope that may allow loss or noise to be minimized when cardiac sound and lung sound are obtained in the interior of the body of a patient.

The technical objects of the inventive concept are not limited to the above-mentioned ones, and the other unmentioned technical objects will become apparent to those skilled in the art from the following description.

According to an embodiment, an esophageal stethoscope may include a cuff, a tube, one end of which is disposed in the interior of the cuff, and extending to a side that is opposite to the one end thereof, a mount member mounted on an opposite end of the tube, and a microphone disposed in the interior of the mount member, wherein the tube includes at least one hole configured to allow sound waves that passes through the cuff to be provided into the tube, and wherein the microphone absorbs cardiac sound and converts the cardiac sound to a cardiac sound electric signal, and is electrically connected to an external device to deliver the cardiac sound electric signal to the external device.

One or more pads may be disposed on an outer peripheral surface of the tube to be spaced apart from each other, and the one or more pads may maintain a shape of the tube.

According to an embodiment, an esophageal stethoscope may include a cuff, a tube, one end of which is disposed in an interior of the cuff, and extending to a side that is opposite to the one end thereof, a microphone disposed in an interior of the tube, a microphone wiring line disposed in the interior of the tube, and one end of which is coupled to the microphone to deliver a cardiac sound electric signal, and a microphone connector coupled to an opposite end of the microphone wiring line, the microphone may absorb cardiac sound and converts the cardiac sound to the cardiac sound electric signal, and may be connected to an external device by the microphone wiring line and the microphone connector to deliver the cardiac sound electric signal to the external device.

The esophageal stethoscope may further include a temperature sensor disposed in the interior of the tube, a compartment may be formed at the one end of the tube, the microphone may be disposed in an interior of the compartment, and the temperature sensor may be disposed outside the compartment of the tube.

A hollow channel connecting the microphone and the one end of the tube may be formed in the tube, and a cross-section of the hollow channel may become smaller toward the microphone.

According to an embodiment, an esophageal stethoscope may include a cuff, a tube, one end of which is disposed in an interior of the cuff, and extending to a side that is opposite to the one end thereof, a first microphone disposed in an interior of the tube, and located at a site that is adjacent to a site, at which a first cardiac sound is generated, when the esophageal stethoscope is inserted, a second microphone disposed in the interior of the tube to be spaced apart from the first microphone, and located at a site that is adjacent to a site, at which a second cardiac sound is generated, when the esophageal stethoscope is inserted, a microphone wiring line disposed in the interior of the tube, and wherein one end of which is coupled to the first microphone and the second microphone to deliver a cardiac sound electric signal, and a microphone connector coupled to an opposite end of the microphone wiring line, the first microphone and the second microphone may absorb cardiac sound and convert the cardiac sound to the cardiac sound electric signal, and may be connected to an external device by the microphone wiring line and the microphone connector to deliver the cardiac sound electric signal to the external device.

The first microphone may absorb the first cardiac sound and may convert the first cardiac sound to a first cardiac sound electric signal, the second microphone may absorb the second cardiac sound and may convert the second cardiac sound to a second cardiac sound electric signal, and the first cardiac sound electric signal and the second cardiac sound electric signal may be used to generate first cardiac sound data and second cardiac sound data, respectively.

The first cardiac sound data and the second cardiac sound data may be used to analyze an insertion location, at which the esophageal stethoscope is inserted.

The analysis of the insertion location may include comparing the obtained cardiac sound data and stored cardiac sound data.

The first microphone or the second microphone may absorb lung sound and converts the lung sound to a lung sound electric signal, the lung sound electric signal may be used to generate lung sound data for analyzing the lung sound, and the analysis of the lung sound may include comparing the obtained lung sound data and lung sound data for each type stored in advance.

Detailed items of the other embodiments are included in the detailed description and the accompanying drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating an esophageal stethoscope according to a first embodiment of the inventive concept;

FIG. 2 is a perspective view illustrating the esophageal stethoscope further including a temperature sensor and a pad according to the first embodiment of the inventive concept;

FIG. 3 is a perspective view illustrating an esophageal stethoscope according to a second embodiment of the inventive concept;

FIG. 4 is a perspective view illustrating the esophageal stethoscope further including a temperature sensor and a pad according to the second embodiment of the inventive concept;

FIG. 5 is a perspective view illustrating the esophageal stethoscope, in which the temperature sensor and a microphone are connected to each other by one wire and a connector, according to the second embodiment of the inventive concept;

FIG. 6 is a perspective view illustrating an esophageal stethoscope according to a third embodiment of the inventive concept;

FIG. 7 is a perspective view illustrating the esophageal stethoscope further including a temperature sensor and a pad according to the third embodiment of the inventive concept;

FIG. 8 is a perspective view illustrating an esophageal stethoscope according to a fourth embodiment of the inventive concept;

FIG. 9 is a perspective view illustrating the esophageal stethoscope further including a temperature sensor and a pad according to the fourth embodiment of the inventive concept; and

FIG. 10 is an exemplary view illustrating a state in which cardiac sound is obtained through the esophageal stethoscope according to the fourth embodiment of the inventive concept.

### DETAILED DESCRIPTION

The above and other aspects, features and advantages of the inventive concept will become apparent from the following description of the following embodiments given in conjunction with the accompanying drawings. However, the inventive concept is not limited by the embodiments disclosed herein but will be realized in various different forms, and the embodiments are provided only to make the disclosure of the inventive concept complete and fully inform the scope of the inventive concept to an ordinary person in the art, to which the inventive concept pertains, and the inventive concept will be defined by the scope of the claims.

The terms used herein are provided to describe the embodiments but not to limit the inventive concept. In the specification, the singular forms include plural forms unless particularly mentioned. The terms “comprises” and/or “comprising” used herein does not exclude presence or addition of one or more other elements, in addition to the aforementioned elements. Throughout the specification, the same reference numerals denote the same elements, and “and/or” includes the respective elements and all combinations of the elements. Although “first”, “second” and the like are used to describe various elements, the elements are not limited by the terms. The terms are used simply to distinguish one element from other elements. Accordingly, it is apparent that a first element mentioned in the following may be a second element without departing from the spirit of the inventive concept.

In the specification, “an external device” (not illustrated) refers to an arbitrary device that may be coupled to a temperature sensor connector 70 or a microphone connector 90 included in an esophageal stethoscope and is connected to a temperature sensor 30 or a microphone 40. For example, the external device may generate data based on an electric signal received from the temperature sensor 30 or the microphone 40, and may visually provide the generated data. To achieve this, the external device may include a controller and a display.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

The terms, such as “below”, “beneath”, “lower”, “above”, and “upper”, which are spatially relative may be used to easily describe a correlation between one element and other elements as illustrated in the drawings. The spatially relative terms have to be understood as terms including different directions of the elements during use or an operation, in addition to the direction illustrated in the drawings. For example, the elements illustrated in the drawings are overturned, the elements “below” or “beneath” another element may be positioned “above” the other element. Accordingly, the term “below” or “beneath” may include “below” or “beneath” and “above”. The element may be oriented in different directions, and accordingly, the spatially relative terms may be construed according to the orientation.

Hereinafter, exemplary embodiments of the inventive concept will be described in detail with reference to the accompanying drawings.

An esophageal stethoscope 100 according to the first embodiment of the inventive concept is an esophageal stethoscope, in which the microphone 40 is disposed in the interior of a mount member 50 mounted on the outside of the esophageal stethoscope 100.

An esophageal stethoscope 200 according to the second embodiment of the inventive concept is an esophageal stethoscope, in which the microphone 40 is disposed between a cuff 10 and a tube 20.

An esophageal stethoscope 300 according to the third embodiment of the inventive concept is an esophageal stethoscope, in which the microphone 40 is disposed in the interior of the tube 20.

An esophageal stethoscope 400 according to the fourth embodiment of the inventive concept is an esophageal stethoscope, in which a first microphone 41 and a second microphone 42 are in the interior of the tube 20.

### First Embodiment

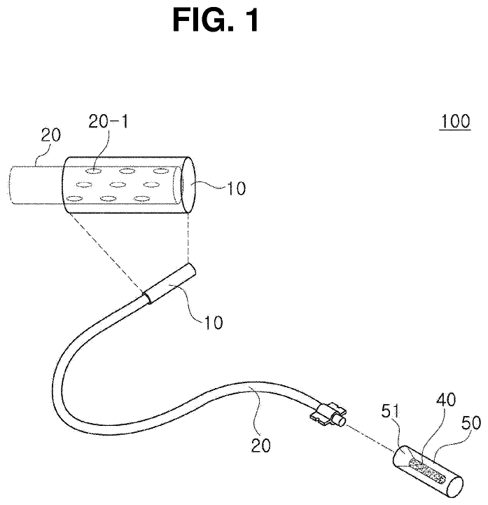

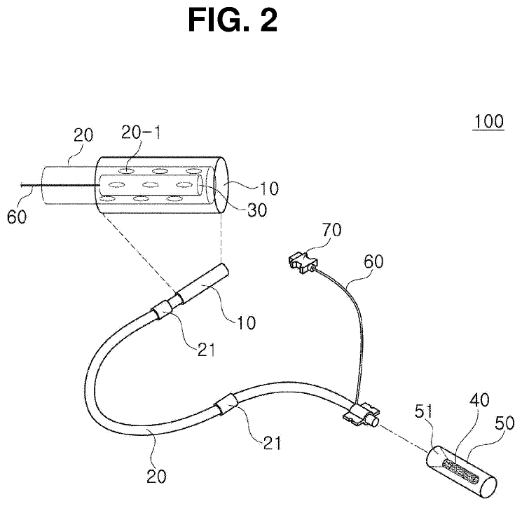

FIG. 1 is a perspective view illustrating an esophageal stethoscope according to a first embodiment of the inventive concept. FIG. 2 is a perspective view illustrating the esophageal stethoscope further including a temperature sensor and a pad according to the first embodiment of the inventive concept.

Hereinafter, the esophageal stethoscope 100 according to the first embodiment of the inventive concept will be described with reference to FIGS. 1 and 2.

Referring to FIG. 1, the esophageal stethoscope 100 according to the first embodiment of the inventive concept includes the cuff 10, the tube 20, the microphone 40, and the mount member 50.

Referring to FIG. 2, the esophageal stethoscope 100 according to the first embodiment of the inventive concept may further include the temperature sensor 30, a temperature sensor wiring line 60, the temperature sensor connector 70, and one or more pads 21.

The cuff 10 is disposed at one end of the tube 20. The cuff 10 is a member that is inserted into the throat of a patient to guide the tube 20. To achieve this, a curved surface may be formed (for smooth insertion) at an end of the cuff 10, and a space for accommodating the tube 20 may be provided in the interior of the cuff 10. That is, the cuff 10 is a member that covers the one end of the tube 20 and stably feeds the tube 20 to the throat of the patient.

As an example, the cuff 10 may be coupled to the tube 20 when the esophageal stethoscope is used, and may be separated from the tube 20 when the esophageal stethoscope is washed after the esophageal stethoscope is used. Through this, the configuration that has been inserted into the body of the patient may be managed more sanitarily after the esophageal stethoscope is used.

The tube 20 is a flexible hollow tubular member that extends from one end to an opposite end thereof. The one end of the tube 20 may be disposed in the interior of the cuff 10, and an opening at the one end of the tube 20 may be blocked by the cuff 10. When the esophageal stethoscope is used, the one end of the tube 20, which is coupled to the cuff 10, is inserted into the body through the oral cavity and the throat of the patient, and the opposite end of the tube 20 is exposed to the outside of the patient.

Cardiac sound and lung sound obtained in the interior of the body of the patient are delivered to the opposite end of the tube 20 through the tube 20. The cardiac sound and the lung sound delivered through the opening at the opposite end of the tube 20 are delivered to the microphone 40 disposed in the interior of the mount member 50.

The one end of the tube 20, which is coupled to the cuff 10, may include a plurality of holes 20-1, through which the cardiac sound and the lung sound generated in the interior of the body of the patient pass. The cardiac sound and the lung sound in the interior of the body of the patient may penetrate the cuff 10 and may be delivered to the interior of the tube 20 through the plurality of holes 20-1 of the tube 20. Through this, the cardiac sound and the lung sound that may penetrate the cuff 10 and are delivered to the interior of the tube 20 through the plurality of holes 20-1 more effectively while the body fluid in the interior of the body of the patient is not introduced into the interior of the tube 20 while being blocked by the cuff 10.

As an example, the one or more pads 21 may be disposed in the tube 20 to be spaced apart from each other in the extension direction of the tube 20 and surround an outer peripheral surface of the tube 20. The one or more pads 21 function to properly maintain the shape of the tube 20 in a process of inserting the tube 20 into the throat of the patient. That is, the one or more pads 21 function to prevent the tube 20 from being folded in the process of inserting the tube 20 into the throat of the patient, and the locations and the number of the pads 21 are not limited.

The temperature sensor 30 may be disposed in the interior of the one end of the tube 20, which is coupled to the cuff 10. That is, the temperature sensor 30 may be disposed in the interior of the tube 20 disposed in the interior of the cuff 10, and may be stably protected from an external force by the cuff 10.

The temperature sensor 30 functions to measure the temperature of the interior of the body of the patient and generate a body temperature electric signal. The temperature sensor 30 may be electrically connected to an external device (not illustrated) by the temperature sensor wiring line 60 to transmit the body temperature electric signal to the external device. Furthermore, the body temperature electric signal may be used to generate body temperature data, and the generated body temperature data may be visually provided to the user through the external device.

To achieve this, one end of the temperature sensor wiring line 60 may be electrically connected to the temperature sensor 30, and may extend in the interior of the tube 20 such that the temperature sensor connector 70 that may be coupled to (docked with) the external device is disposed at an opposite end of the temperature sensor wiring line 60. The external device may receive the body temperature electric signal, and may generate body temperature data based on the received electric signal. Furthermore, the external device may visualize the generated body temperature data and may provide the visualized body temperature data to a user in real time.

The microphone 40 is accommodated in (disposed in the interior of) the mount member 50, and is located adjacent to the opposite end of the tube 20. To achieve this, an accommodation space that accommodates the microphone 40 may be provided in the interior of the mount member 50, and the mount member 50 may be mounted on the opposite end of the tube 20.

The microphone 40 may be connected to the opposite end of the tube 20 by a hollow channel 51 formed in the mount member 50. In this case, at least a portion of the opposite end of the tube 20 may be inserted into the hollow channel 51 of the mount member 50. The microphone 40 may be directly connected to the opening at the opposite end of the tube 20 through the hollow channel 51 of the mount member 50 while being blocked from the outside. Through this, in the esophageal stethoscope 100 according to the first embodiment of the inventive concept, the cardiac sound and the lung sound delivered through the tube 20 may be delivered to the microphone 40 while they are not leaked to the outside and their losses are minimized.

As an example, the hollow channel 51 of the mount member 50 may have a tapered shape, the cross-section of which becomes smaller toward the microphone. The opposite end of the tube 20 may be adhered and coupled to the hollow channel 51 of the mount member 50 more tightly. Through this, the cardiac sound and the lung sound delivered through the opening of the opposite end of the tube 20 may be concentrated in the microphone 40 with no loss.

Like the microphone 40 included in the esophageal stethoscope 200 according to the second embodiment of the inventive concept, the microphone 40 included in the esophageal stethoscope 300 according to the third embodiment of the inventive concept, and the first microphone 41 and the second microphone 42 included in the esophageal stethoscope 400 according to the fourth embodiment of the inventive concept, which will be described below, the microphone 40 included in the esophageal stethoscope 100 according to the first embodiment of the inventive concept also may convert the cardiac sound or lung sound to a cardiac sound electric signal or a lung sound electric signal and may be electrically connected to the external device by the microphone wiring line and the microphone connector to deliver the cardiac sound electric signal or the lung sound electric signal to the external device.

Furthermore, the cardiac sound/lung sound electric signal is used to generate cardiac sound/lung sound data for analyzing the cardiac sound/lung sound. The external device may analyze and visualize the generated cardiac sound/lung sound data and provide the analyzed and visualized cardiac sound/lung sound data to the user in real time. In a detailed example, when both the cardiac sound and the lung sound are obtained, the cardiac sound and the lung sound may be separated based on their respective frequency area features. That is, the microphone 40 and the external device that received an electric single from the microphone 40 may separate the cardiac sound and the lung sound obtained simultaneously based on the respective frequency area features, and may use the separated cardiac sound and lung sound in the analysis of the cardiac sound/lung sound.

Furthermore, when the temperature sensor 30 is further included, the temperature sensor wiring line 60 and the microphone wiring line 80 may be formed of one connected wiring line, and the temperature sensor connector 70 and the microphone connector 90 also may be formed of one connector and may be connected to the external device.

### Second Embodiment

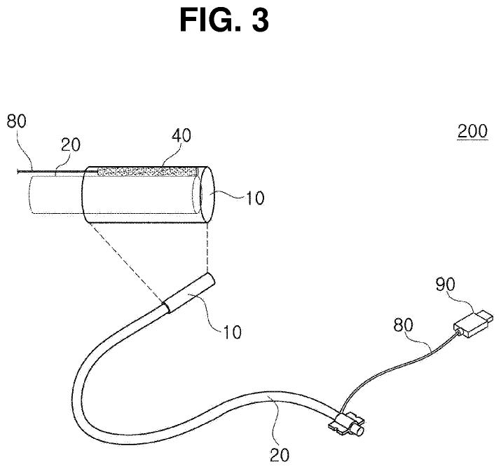

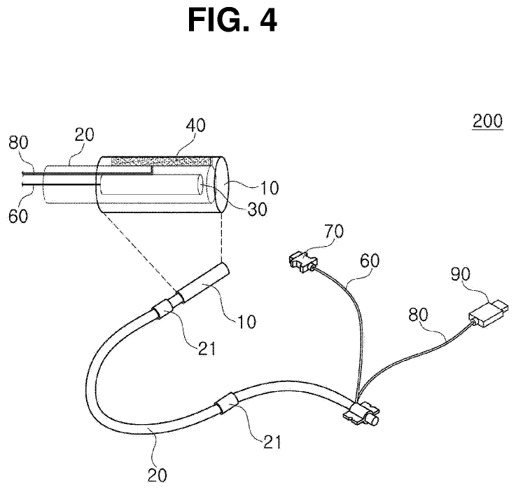

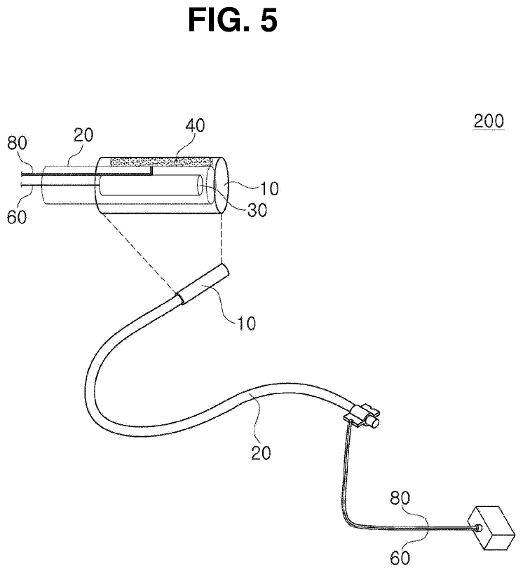

FIG. 3 is a perspective view illustrating an esophageal stethoscope according to a second embodiment of the inventive concept. FIG. 4 is a perspective view illustrating the esophageal stethoscope further including a temperature sensor and a pad according to the second embodiment of the inventive concept. FIG. 5 is a perspective view illustrating the esophageal stethoscope, in which the temperature sensor and a microphone are connected to each other by one wire and a connector, according to the second embodiment of the inventive concept.

Hereinafter, the esophageal stethoscope 200 according to the second embodiment of the inventive concept will be described with reference to FIGS. 3 to 5.

Referring to FIG. 3, the esophageal stethoscope 200 according to the second embodiment of the inventive concept includes the cuff 10, the tube 20, the temperature sensor 30, the microphone 40, the microphone wiring line 80, and the microphone connector 90.

Referring to FIG. 4, the esophageal stethoscope 200 according to the second embodiment of the inventive concept may further include the temperature sensor 30, the temperature sensor wiring line 60, the temperature sensor connector 70, and the one or more pads 21.

The cuff 10, the tube 20, the temperature sensor 30, the temperature sensor wiring line 60, the temperature sensor connector 70, and the external device according to the first embodiment of the inventive concept may be similarly applied to the cuff 10, the tube 20, the temperature sensor 30, the temperature sensor wiring line 60, the temperature sensor connector 70, and the external device according to the second embodiment of the inventive concept. A repeated description of the above-mentioned contents will be omitted.

In the esophageal stethoscope 200 according to the second embodiment of the inventive concept, the microphone 40 is disposed between the cuff 10 and the tube 20. That is, in the esophageal stethoscope 200 according to the second embodiment of the inventive concept, the microphone 40 may be inserted into the interior of the body of the patient together with the cuff 10, and the cardiac sound and the lung sound generated in the interior of the body of the patient may penetrate the cuff 10 and may be directly delivered. As a result, in the esophageal stethoscope 200 according to the second embodiment of the inventive concept, because the cardiac sound and the lung sound may be delivered to the microphone 40 even though they do not penetrate the tube 20, losses or noise generated in the delivery process may be decreased. Furthermore, a hole does not need to be formed at the one end of the tube 20 as in the esophageal stethoscope 100 according to the first embodiment of the inventive concept, and the opening at the opposite end of the tube 20 may be closed. This is because the cardiac sound and the lung sound of the patient penetrate the cuff 10 and are directly delivered to the microphone 40.

The microphone 40 converts the cardiac sound or lung sound to a cardiac sound electric signal or a lung sound electric signal. The cardiac sound/lung sound electric signal is used to generate cardiac sound data or lung sound data for analyzing the cardiac sound/lung sound.

The microphone 40 may be electrically connected to an external device (not illustrated) by the microphone wiring line 80 and the microphone connector 90, and may transmit the cardiac sound/lung sound electric signal to the external device.

One end of the microphone wiring line 80 is coupled to the microphone 40 between the cuff 10 and the tube 20. The microphone wiring line 80 may extend to the interior of the tube 20, and an opposite end of the microphone wiring line 80 may be exposed to the outside of the tube 20. Furthermore, the opposite end of the microphone wiring line 80 is coupled to the microphone connector 90 that may be coupled to (docked with) the external device.

The microphone connector 90 is a connection member that is formed in an arbitrary form. That is, the shape of the microphone connector is not limited, and the microphone connector may include a connector of a standard used conventionally or a connector of a unique shape, which is different from the standard. As a detailed example, when the microphone connector 90 has a form that is different from the standard, the esophageal stethoscope including the corresponding microphone connector 90 may be used only when it is connected to a device including a connection terminal that may accommodate the shape of the microphone connector 90 to be coupled.

The external device may receive the cardiac sound/lung sound electric signal from the microphone 40 through the microphone wiring line 80 and the microphone connector 90, and may generate cardiac sound/lung sound data based on the received cardiac sound/lung sound electric signal. The external device may analyze and visualize the generated cardiac sound/lung sound data and provide the analyzed and visualized cardiac sound/lung sound data to the user in real time.

FIG. 5 is a perspective view illustrating the esophageal stethoscope, in which the temperature sensor and a microphone are connected to each other by one wire and a connector, according to the second embodiment of the inventive concept.

Referring to FIG. 5, when the esophageal stethoscope further includes the temperature sensor 30, the temperature sensor wiring line 60 and the microphone wiring line 80 may be formed of one connected wiring line (not illustrated), or may be coupled to the temperature sensor wiring line 60 and the microphone wiring line 80 to be connected to one connector. That is, the temperature sensor connector and the microphone connector may not be separately provided but may be formed of one connector and be connected to the external device. Then, the external device may be electrically connected to the temperature sensor 30 and the microphone 40 by one connector, and may process data received from the temperature sensor 30 and the microphone 40, respectively. That is, the one external device may be connected to both of the temperature sensor 30 and the microphone 40 by the one connector to analyze the body temperature data and the cardiac sound/lung sound data. Through this, costs of the manufacturing process may be reduced, and the body temperature data and the cardiac sound/lung sound data may be complexly analyzed only with the one external device so that it is not necessary to provide a plurality of external devices (external device for respective analysis target data).

### Third Embodiment

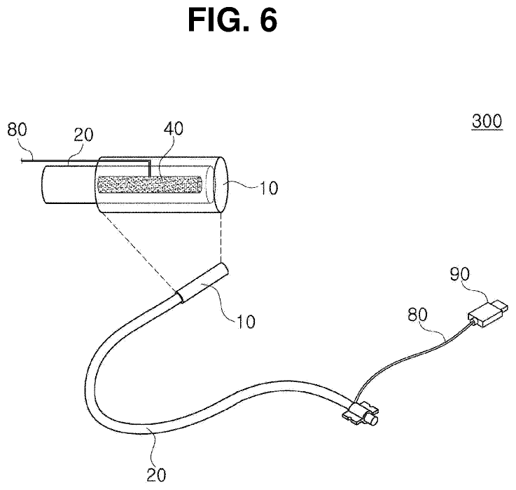

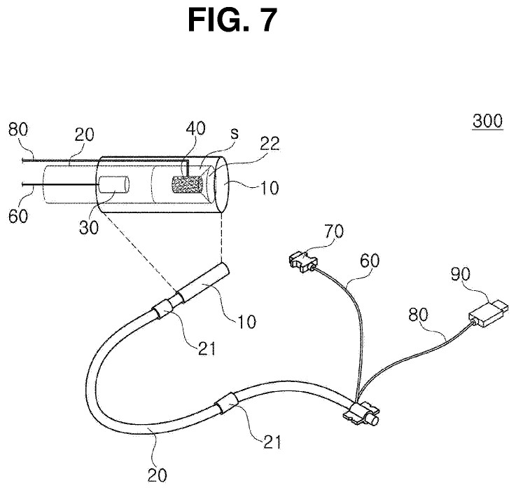

FIG. 6 is a perspective view illustrating an esophageal stethoscope according to a third embodiment of the inventive concept. FIG. 7 is a perspective view illustrating the esophageal stethoscope further including a temperature sensor and a pad according to the third embodiment of the inventive concept.

Hereinafter, the esophageal stethoscope 300 according to the third embodiment of the inventive concept will be described with reference to FIGS. 6 and 7.

Referring to FIG. 6, the esophageal stethoscope 300 according to the third embodiment of the inventive concept includes the cuff 10, the tube 20, the microphone 40, the microphone wiring line 80, and the microphone connector 90.

Referring to FIG. 7, the esophageal stethoscope 300 according to the third embodiment of the inventive concept may further include the temperature sensor 30, the temperature sensor wiring line 60, the temperature sensor connector 70, and the one or more pads 21.

The cuff 10, the tube 20, the pad 21, the temperature sensor 30, the temperature sensor wiring line 60, the temperature sensor connector 70, the microphone wiring line 80, the microphone connector 90, and the external device according to the second embodiment of the inventive concept may be similarly applied to the cuff 10, the tube 20, the pad 21, the temperature sensor 30, the temperature sensor wiring line 60, the temperature sensor connector 70, the microphone wiring line 80, the microphone connector 90, and the external device according to the third embodiment of the inventive concept. A repeated description of the above-mentioned contents will be omitted.

In the esophageal stethoscope 300 according to the third embodiment of the inventive concept, the microphone 40 is disposed in the interior of the one end of the tube 20. Furthermore, when the temperature sensor 30 is further included, the temperature sensor 30 and the microphone 40 may be disposed to be adjacent to each other or be spaced apart from each other. That is, the temperature sensor 30 and the microphone 40 may be disposed in the interior of the tube 20 disposed in the interior of the cuff 10, and may be stably protected from an external force by the cuff 10.

As an example, a compartment “s” for accommodating the microphone 40 may be formed at the one end of the tube 20. That is, the microphone 40 may be disposed in the interior of the compartments “s” of the tube 20, and the temperature sensor 30 may be disposed outside the compartment “s” of the tube 20. Accordingly, the temperature sensor 30 and the microphone 40 may be provided in completely independent spaces to measure the body temperature and absorb the cardiac sound/lung sound, respectively, while not interrupting each other

As another example, a hollow channel 22 connecting the microphone 40 and the one end (may be the opening at the one end) of the tube 20 may be formed in the tube 20. Accordingly, because the microphone 40 may be disposed in the interior of the tube 20 to be stably protected and may be disposed to directly contact the cuff 10, the cardiac sound or the lung sound that penetrates the cuff 10 may be directly delivered and the losses of the cardiac sound and the lung sound may be minimized.

As another example, the cross-sectional area of the hollow channel 22 of the tube 20 may have a shape that becomes smaller toward the microphone 40. Through this, the cardiac sound or the lung sound may be concentrated more in the microphone 40 and be delivered to minimize generation of noise.

Meanwhile, in the esophageal stethoscope 300 according to the third embodiment of the inventive concept, the microphone 40 also may be electrically connected to the external device (not illustrated) by the microphone wiring line 80 and the microphone connector 90. One end of the microphone wiring line 80 may be connected to the microphone 40, and may extend in the interior of the tube 20 and the opposite end may be exposed to the outside of the tube 20. Furthermore, the opposite end of the microphone wiring line 80 is coupled to the microphone connector 90 that may be coupled to (docked with) the external device.

Furthermore, unlike the microphone wiring line 80 of the second embodiment of the inventive concept, the one end of the microphone wiring line 80 of the third embodiment of the inventive concept may be directly connected to the microphone 40 disposed in the interior of the tube 20 while not passing through the tube 20 to be connected to the microphone 40 between the cuff 10 and the tube 20. That is, in the esophageal stethoscope 300 according to the third embodiment of the inventive concept, the microphone wiring line 80 does not extend while passing through the tube 20. Accordingly, efforts and costs for the manufacturing process for disposing a conductive line can be reduced.

Meanwhile, as in FIG. 5 of the second embodiment, when the esophageal stethoscope further includes the temperature sensor 30, the temperature sensor wiring line 60 and the microphone wiring line 80 may be formed of one connected wiring line, or may be coupled to the temperature sensor wiring line 60 and the microphone wiring line 80 to be connected to one connector. That is, the temperature sensor connector and the microphone connector may not be separately provided but may be formed of one connector and be connected to the external device. Then, the external device may be electrically connected to the temperature sensor 30 and the microphone 40 by one connector, and may process data received from each of the temperature sensor 30 and the microphone 40. That is, the one external device may be connected to both of the temperature sensor 30 and the microphone 40 by the one connector to analyze the body temperature data and the cardiac sound/lung sound data. Through this, costs of the manufacturing process may be reduced, and the body temperature data and the cardiac sound/lung sound data may be complexly analyzed only with the one external device so that it is not necessary to provide a plurality of external devices (external device for respective analysis target data).

### Fourth Embodiment

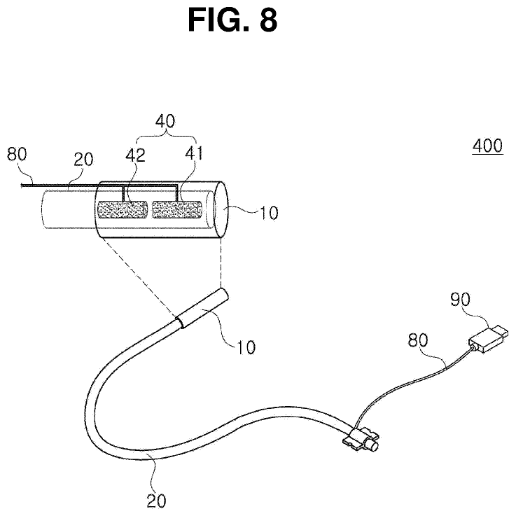

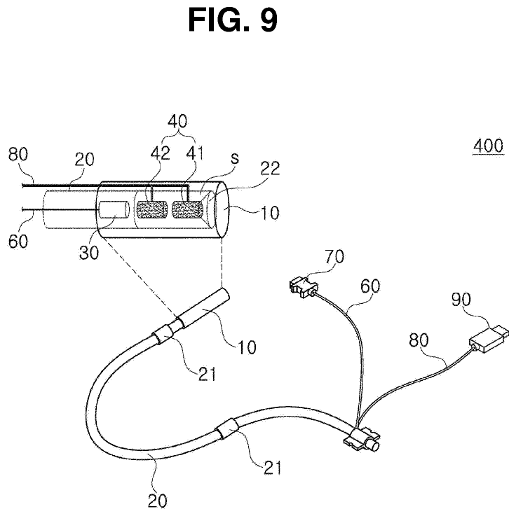

FIG. 8 is a perspective view illustrating an esophageal stethoscope according to a fourth embodiment of the inventive concept. FIG. 9 is a perspective view illustrating the esophageal stethoscope further including a temperature sensor and a pad according to the fourth embodiment of the inventive concept.

Hereinafter, the esophageal stethoscope 400 according to the fourth embodiment of the inventive concept will be described with reference to FIGS. 8 and 9.

Referring to FIG. 8, the esophageal stethoscope 400 according to the fourth embodiment of the inventive concept includes the cuff 10, the tube 20, the first microphone 41, the second microphone 42, the microphone wiring line 80, and the microphone connector 90.

Referring to FIG. 9, the esophageal stethoscope 400 according to the fourth embodiment of the inventive concept may further include the temperature sensor 30, the temperature sensor wiring line 60, the temperature sensor connector 70, and the one or more pads 21.

The cuff 10, the tube 20, the pad 21, the temperature sensor 30, the temperature sensor wiring line 60, the temperature sensor connector 70, the microphone wiring line 80, the microphone connector 90, and the external device according to the second embodiment of the inventive concept may be similarly applied to the cuff 10, the tube 20, the pad 21, the temperature sensor 30, the temperature sensor wiring line 60, the temperature sensor connector 70, the microphone wiring line 80, the microphone connector 90, and the external device according to the fourth embodiment of the inventive concept. A repeated description of the above-mentioned contents will be omitted.

In the esophageal stethoscope 400 according to the fourth embodiment of the inventive concept, the first microphone 41 and the second microphone 42 are disposed in the interior of the one end of the tube 20. The first microphone 41 functions to obtain a first cardiac sound and the second microphone 42 functions to obtain a second cardiac sound.

“Cardiac sound” is sound that is generated when the heart beats to send blood to the entire body. Normal cardiac sound is classified into “a first cardiac sound” and “a second cardiac sound”. The heart includes four chambers called the left atrium, the left ventricle, the right atrium, and the right ventricle. Valves are located between the chambers, and the valves function to help the blood flow only in one direction. The blood that returned from the pulmonary vein and the vena cava to the heart flows into the ventricles as the atriums are contracted, and the first cardiac sound is generated while the mitral valve and the tricuspid valve are closed as the left atrium and the left ventricle are contracted. The second cardiac sound is generated while the aortic valve and the pulmonary valve are closed to prevent the blood from flowing reversely when the ventricle that has been completely contracted starts to be released after the blood is pumped out to the main artery and the pulmonary artery while the aortic valve and the pulmonary valve are opened. While the above-mentioned process is repeated, the first cardiac sound and the second cardiac sound are alternately generated. Meanwhile, generally, the first cardiac sound is a relatively low sound that is heard around the apical region, and the second cardiac sound is a relatively high sound that is heard in the base.

In the esophageal stethoscope 400 according to the fourth embodiment of the inventive concept, the first microphone 41 is disposed in the interior of the tube 20, and is located at a site that is adjacent to the site, at which the first cardiac sound is generated, when the esophageal stethoscope 400 is inserted. The second microphone 42 is disposed in the interior of the tube 20 to be spaced apart from the first microphone 41, and is disposed at a site that is adjacent to a site, at which the second cardiac sound is generated, when the esophageal stethoscope 400 is inserted. That is, the first microphone 41 and the second microphone 42 are disposed to be spaced apart from each other by a specific distance to be located at sites that are adjacent to the sites, at which the first cardiac sound and the second cardiac sound are generated, when the esophageal stethoscope 400 is inserted into the body through the throat of the patient. Then, the “specific distance” may be an interval between the sites of the heart, at which the first cardiac sound and the second cardiac sound are generated most greatly. Through this, the first microphone 41 and the second microphone 42 may obtain the sound waves for the first cardiac sound and the second cardiac sound with intensively strong amplitudes, respectively.

When one microphone obtains cardiac sound to distinguish the first cardiac sound and the second cardiac sound, the sizes of the first cardiac sound and the second cardiac sound may be obtained while being distorted as the distance from the inserted esophageal stethoscope and the microphones become different due to the difference between the sites, at which the first cardiac sound and the second cardiac sound are generated. That is, if the microphone is located adjacent to the site, at which the first cardiac sound is generated, it may look as if the second cardiac sound is smaller than its actual size. Unlike this, if two microphones are located at sites that are adjacent to the sites, at which the first cardiac sound and the second cardiac sound are generated, respectively, as in the fourth embodiment of the inventive concept, the cardiac sound data for the first cardiac sound and the second cardiac sound may be obtained more precisely. Accordingly, it may be determined whether the first cardiac sound and the second cardiac sound are abnormal, and a precise diagnosis may be made for a patient, whose any one of a heart portion that generates the first cardiac sound (for example, the mitral valve and the tricuspid valve) or a heart portion that generates the second cardiac sound (for example, the aortic valve and the pulmonary valve) is abnormal.

The first microphone 41 and the second microphone 42 absorb the cardiac sound and convert the absorbed cardiac sound to a cardiac sound electric signal, and is connected to the external device by the microphone wiring line 80 and the microphone connector 90 to deliver the cardiac sound electric signal to the external device. As a detailed example, the first microphone 41 absorbs the first cardiac sound and converts the absorbed first cardiac sound to a first cardiac sound electric signal, and the second microphone 42 absorbs the second cardiac sound and converts the absorbed second cardiac sound to a second cardiac sound electric signal. The first cardiac sound electric signal and the second cardiac sound electric signal, which have been obtained, are used to generate first cardiac sound data and second cardiac sound data. The first cardiac sound data and the second cardiac sound data may be analyzed and visualized by the external device and may be provided to the user in real time.

As an example, the first microphone 41 and the second microphone 42 may absorb lung sound and convert the lung sound to a lung sound electric signal. That is, the first microphone 41 and the second microphone 42 may absorb the cardiac sound and the lung sound simultaneously. The cardiac sound and the lung sound absorbed simultaneously may be separated (for example, separated based on their frequency area features) and converted to a cardiac sound electric signal and a lung sound electric signal, and may be used to generate the cardiac sound/lung sound data for analyzing the cardiac sound/lung sound.

Meanwhile, the analysis of the lung sound includes compring the lung sound data and the lung sound data for types stored in advance to provide additional information. Because the lung sound is generally generated when an inspiratory organ has a problem, the data classified for the types may be stored in advance according to the causes of the lung sound, the lung sound data stored in advance may be compared with the lung sound data for analysis, and the cause of the abnormality may be analyzed.

As another example, the cardiac sound data may be used to analyze the insertion location of the esophageal stethoscope. That is, the first cardiac sound data and the second cardiac sound data absorbed and obtained by the first microphone 41 and the second microphone 42 may be used to analyze the insertion location (the insertion depth) of the esophageal stethoscope. As a detailed example, the analysis of the cardiac sound may include comparing the cardiac sound data stored in advance and the cardiac sound data and the information on the insertion location may be additionally provided. It may be analyzed whether the inserted esophageal stethoscope is properly located by storing the first cardiac sound data and the second cardiac sound data in advance and managing the first cardiac sound data and the second cardiac sound data when the first microphone 41 and the second microphone 42 are precisely located at sites, at which the first cardiac sound and the second cardiac sound are generated, and by comparing the first cardiac sound data and the second cardiac sound data, which have been obtained with the data stored in advance.

The microphone wiring line 80 may be connected to the first microphone 41 and the second microphone 42 by one wiring line, and may include a plurality of wiring lines to be connected to the first microphone 41 and the second microphone 42, respectively. Furthermore, as another example, when the temperature sensor 30 is further included, the microphone wiring line 80 may be a separate microphone wiring line 80 from the temperature sensor wiring line 60 or the temperature sensor 30, the first microphone 41, and the second microphone 42 may be connected to each other by one wiring line.

One end of the microphone wiring line 80 is coupled to the first microphone 41 and the second microphone 42 to deliver the cardiac sound electric signal, and an opposite end of the microphone wiring line 80 is coupled to the microphone connector 90. The microphone connector 90 is coupled to (docked with) the external device, and functions to directly connect the first microphone 41 and the second microphone 42 to the external device.

The microphone connector 90 is a connection member that is formed in an arbitrary form as described in the second embodiment. That is, the shape of the microphone connector 90 is not limited, and the microphone connector may include a connector of a standard used conventionally or a connector of a unique shape, which is different from the standard.

Meanwhile, as in FIG. 5 of the second embodiment, when the esophageal stethoscope further includes the temperature sensor 30, the temperature sensor wiring line 60 and the microphone wiring line 80 may be formed of one connected wiring line, or the temperature sensor wiring line 60 and the microphone wiring line 80 may be coupled to each other to be connected to one connector. That is, the temperature connector and the microphone connector may not be separately provided but may be formed of one connector and be connected to the external device. Then, the external device may be electrically connected to the temperature sensor 30, the first microphone 41, and the second microphone 42 simultaneously by one connector, and may process data received from each of the temperature sensor 30, the first microphone 41, and the second microphone 42. That is, the one external device may be connected to all of the temperature sensor 30, the first microphone 41, and the second microphone 42 by the one connector to analyze the body temperature data and the cardiac sound/lung sound data. Through this, costs of the manufacturing process may be reduced, and the body temperature data and the cardiac sound/lung sound data may be complexly analyzed only with the one external device so that it is not necessary to provide a plurality of external devices (external device for respective analysis target data).

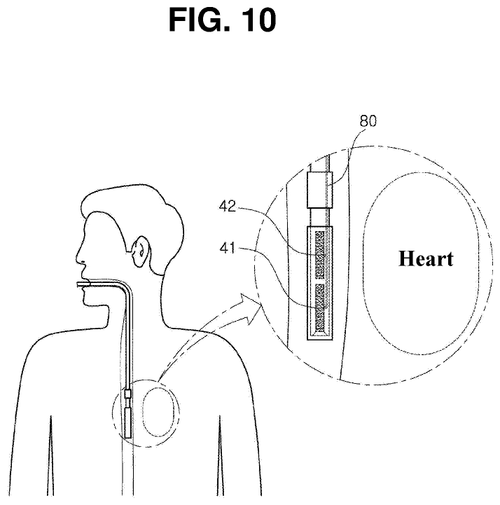

FIG. 10 is an exemplary view illustrating a state in which cardiac sound is obtained through the esophageal stethoscope according to the fourth embodiment of the inventive concept.

Referring to FIG. 10, portions of the cuff 10 and the tube 20 of the esophageal stethoscope are inserted into the interior of the body of the patient through the throat of the patient. The cuff 10 portion is inserted to a point that is adjacent to the heart. The first microphone 41 and the second microphone 42 disposed in the interior of the tube 20 is located to adjacent to the points, at which the first cardiac sound and the second cardiac sound are generated, respectively, to absorb the first cardiac sound and the second cardiac sound generated as the heart beats, convert the absorbed first cardiac sound and second cardiac sound to electric signals, and deliver the electric signal to the external device (not illustrated) coupled to the microphone connector 90 through the microphone wiring line 80. The delivered cardiac sound electric signal is used to generate cardiac sound data for analysis of cardiac sound.

The steps of a method or an algorithm that have been described in relation to the embodiments of the inventive concept may be directly implemented by hardware, may be implemented by a software module executed by hardware, or may be implemented by a combination thereof. The software module may reside in a random access memory (RAM), a read only memory (ROM), an erasable programmable ROM (EPROM), an electrically erasable programmable ROM (EEPROM), a flash memory, a hard disk, a detachable disk, a CD-ROM, or a computer readable recording medium in an arbitrary form, which is well known in the art to which the inventive concept pertains.

According to the inventive concept, cardiac sound and lung sound obtained in the interior of the body of a patient may be visually provided not by allowing the cardiac sound and the lung sound to be directly heard through a stethoscope but by using an electric signal obtained through conversion by directly connecting a microphone included in the esophageal stethoscope to an external device. Accordingly, a user may monitor a vital sign of a patient more precisely in real time before and after a surgical operation or during a surgical operation. Furthermore, according to the inventive concept, an esophageal stethoscope or a microphone included in the esophageal stethoscope is directly connected to an external device to provide information on cardiac sound and lung sound.

In more detail, because cardiac sound and lung sound may be provided in a form of an electric signal, to which the cardiac sound and the lung sound are converted by a microphone connector that may be directly connected to a microphone wiring line disposed in the interior of a tube and an external device, loss of sound or noise can be minimized so that high-quality information on the cardiac sound and the lung sound can be obtained.

The effects of the inventive concept are not limited thereto, and other unmentioned effects of the inventive concept may be clearly appreciated by those skilled in the art from the following descriptions.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. An esophageal stethoscope comprising:
a cuff;
a tube, one end of which is disposed in the interior of the cuff, and extending to a side that is opposite to the one end thereof;
a mount member mounted on an opposite end of the tube; and
a microphone disposed in the interior of the mount member,
wherein the tube includes at least one hole configured to allow sound waves that passes through the cuff to be provided into the tube, and
wherein the microphone absorbs cardiac sound and converts the cardiac sound to a cardiac sound electric signal, and is electrically connected to an external device to deliver the cardiac sound electric signal to the external device.

2. The esophageal stethoscope of claim 1, wherein one or more pads are disposed on an outer peripheral surface of the tube to be spaced apart from each other, and
wherein the one or more pads maintain a shape of the tube.

3. An esophageal stethoscope comprising:
a cuff;
a tube, one end of which is disposed in an interior of the cuff, and extending to a side that is opposite to the one end thereof;
a microphone disposed in an interior of the tube;
a microphone wiring line disposed in the interior of the tube, and one end of which is coupled to the microphone to deliver a cardiac sound electric signal; and
a microphone connector coupled to an opposite end of the microphone wiring line,
wherein the microphone absorbs cardiac sound and converts the cardiac sound to the cardiac sound electric signal, and is connected to an external device by the microphone wiring line and the microphone connector to deliver the cardiac sound electric signal to the external device.

4. The esophageal stethoscope of claim 3, further comprising:
a temperature sensor disposed in the interior of the tube,
wherein a compartment is formed at the one end of the tube,
wherein the microphone is disposed in an interior of the compartment, and
wherein the temperature sensor is disposed outside the compartment of the tube.

5. The esophageal stethoscope of claim 4, wherein a hollow channel connecting the microphone and the one end of the tube is formed in the tube, and
wherein a cross-section of the hollow channel becomes smaller toward the microphone.

6. An esophageal stethoscope comprising:
a cuff;
a tube, one end of which is disposed in an interior of the cuff, and extending to a side that is opposite to the one end thereof;
a first microphone disposed in an interior of the tube, and located at a site that is adjacent to a site, at which a first cardiac sound is generated, when the esophageal stethoscope is inserted;
a second microphone disposed in the interior of the tube to be spaced apart from the first microphone, and located at a site that is adjacent to a site, at which a second cardiac sound is generated, when the esophageal stethoscope is inserted;
a microphone wiring line disposed in the interior of the tube, and wherein one end of which is coupled to the first microphone and the second microphone to deliver a cardiac sound electric signal; and
a microphone connector coupled to an opposite end of the microphone wiring line,
wherein the first microphone and the second microphone absorb cardiac sound and convert the cardiac sound to the cardiac sound electric signal, and are connected to an external device by the microphone wiring line and the microphone connector to deliver the cardiac sound electric signal to the external device.

7. The esophageal stethoscope of claim 6, wherein the first microphone absorbs the first cardiac sound and converts the first cardiac sound to a first cardiac sound electric signal,
wherein the second microphone absorbs the second cardiac sound and converts the second cardiac sound to a second cardiac sound electric signal, and
wherein the first cardiac sound electric signal and the second cardiac sound electric signal are used to generate first cardiac sound data and second cardiac sound data, respectively.

8. The esophageal stethoscope of claim 7, wherein the first cardiac sound data and the second cardiac sound data are used to analyze an insertion location, at which the esophageal stethoscope is inserted.

9. The esophageal stethoscope of claim 8, wherein the analysis of the insertion location includes:
comparing the obtained cardiac sound data and stored cardiac sound data.

10. The esophageal stethoscope of claim 7, wherein the first microphone or the second microphone absorbs lung sound and converts the lung sound to a lung sound electric signal,
wherein the lung sound electric signal is used to generate lung sound data for analyzing the lung sound, and
wherein the analysis of the lung sound includes:
comparing the obtained lung sound data and lung sound data for each type stored in advance.
